(19) United States
(12) Patent Application Publication
(10) Pub. No.: US 2023/0238110 A1
(43) Pub. Date: Jul. 27, 2023

(54) METHOD FOR GENERATING INDIVIDUAL NUTRITIONAL RECOMMENDATIONS FOR A USER

(71) Applicant: Viktor Nikolaevich LITUEV, Moscow (RU)

(72) Inventor: Viktor Nikolaevich LITUEV, Moscow (RU)

(21) Appl. No.: 18/020,921

(22) PCT Filed: Jul. 17, 2021
(86) PCT No.: PCT/RU2021/050223

§ 371 Date: Feb. 10, 2023

(30) Foreign Application Priority Data
Aug. 12, 2020 (RU) 2020127074

(51) Int. Cl. G16H 20/60; G06Q 30/0601; G06F 17/40

(52) U.S. Cl. CPC G16H 20/60; G06Q 30/0631; G06F 17/40

(57) ABSTRACT

The invention relates to the field of dietetics, and more specifically to methods for generating individual nutritional recommendations for a user, comprising the following steps: data on at least one food product and/or meal of a user are obtained; and at least one recommendation is generated for the user. The present solution has developed digital technology for accurately measuring and diagnosing the interaction of organic and inorganic elements of food products, drugs, parameters of blood tests and pathologies on the basis of statistical methods.

### TECHNICAL FIELD OF INVENTION

The invention relates to the field of dietetics, and more specifically to methods for generating individual nutritional recommendations for a user, comprising the following steps: data on at least one food product and/or meal of a user are obtained; and at least one recommendation is generated for the user.

The following terms and definitions are used in this description:

Permissible (reference) values is a medical term used for laboratory and clinical tests; meaning ranges of specific clinical and biochemical values obtained during mass population studies and corresponding to the values characteristic of a healthy adult. Examples of reference values (ranges): hemoglobin: 120-150 g/L; glucose: 3.9-5.8 mmol/L; platelets: 150-370 U9/L. Most reference values are expressed in different units of measurement.

Cluster analysis is a multivariate statistical procedure that collects data containing information about object sampling and dividing the objects into relatively homogenous groups.

Factor analysis is a multivariate method used for studying the relations between variable values. It is assumed that known variables are less dependent on the number of unknown variables and unbiased error.

Multiple regression is a statistical method which is an extended version of simple regression that allows for making predictions and drawing conclusions from latent states relative, for example, to one dependent variable and based on the changes and actions of two or more independent variables. Individual patient measurements can be such dependent and independent variables. If the regression equation comprises default values, relative weights or contributions of each independent (predicting) variable to the change in the dependent variable.

### PRIOR ART

It is hard to find an area of human activity unaffected by food. An understandable effect of food is a change in blood chemistry that, in turn, leads to the emergence or disappearance of a number of pathologies. Therefore, there can be different approaches in generating individual nutritional recommendations for a user.

A known example of prior art is a method for generating individual nutritional recommendations for a user performed by at least one processor, wherein: data on at least one food product and/or meal of a user are obtained; and at least one recommendation is generated for the user, see RF patent No 2721234, published in 2020.

The above method is the closest to the essence and the technical result of the claimed invention and is assumed herein as a prototype of the claimed invention.

The drawback of the above prototype is insufficient precision of generating individual nutritional recommendations for a user to improve their health due to not accounting for statistical relation of food parameters and individual user parameters.

### DISCLOSURE OF THE INVENTION

The present invention relies on this novel observation with the primary aim to offer a method for generating individual nutritional recommendations for a user performed by at least one processor that ensures improved precision of generating individual nutritional recommendations for a user to improve their health, said objective being the claimed technical objective of the present invention.

For this purpose
parameters characterizing the user's condition are measured including complete blood count, pathologies are identified, and individual user data is converted into a numerical sequence,
each product is assigned a corresponding numerical sequence depending on the individual nutrient levels,
correlations between individual user parameters and food parameters are calculated by designing a personal matrix of each product effect on the user parameters; for this purpose, factorial weights of each individual product effect on each user parameter are calculated,
the resulting personal matrix of each product effect on the user parameters comprising factorial weights of each individual product effect on the user pathology is statistically processed using the processor; the matrix is further employed to generate recommendations for the user, wherein products with negative factorial weights are not recommended and products with positive factorial weights are recommended.

These useful features make it possible to improve precision of generating individual nutritional recommendations for a user to improve their health.

In order to obtain relevant results when diagnosing the interaction between organic and inorganic food components, medicines, blood test data, and pathology data, a digital database must conform to the following requirements.

Firstly, an informational component or parameters must have qualitative individuality expressed as digital combinations of binaries.

Secondly, as all components and parameters are expressed in different units (grams, milligrams, micrograms, grams per liter, etc), they must be comparable. Comparability of al components and parameters can be ensured by converting the data into probabilistic form via a system of binaries.

Thirdly, the components and parameters selected for analysis must have an adequate statistical form. In this case, the most suitable statistical form is mass data matrix that has a number of commonly accessible and effective statistical analysis methods.

The claimed method relies on generating a personal matrix of each product effect on the user parameters, wherein all values are numerical, which means that they are comparable and can be processed by mathematical methods because the issue of incompatibility due to difference in dimensions is eliminated. It creates an opportunity to find correlations between the parameters that could not be compared before. Such correlations, in turn, allow for determining factorial weights of individual product effect on the user's blood parameters and, therefore, on the user's pathology itself.

This is the basis for generating recommendations for the user, wherein products with negative factorial weights are not recommended and products with positive factorial weights are recommended.

A preferred version of the invention includes a method, wherein the cells of the personal matrix contain the values used in relation to each measured parameter divided into six ranges, namely:
i the value of the measured parameter falls within the range of permissible values for said parameter;
ii the value of the measured parameter is more than the average value of the range of permissible values;
iii the value of the measured parameter is less than the average value of the range of permissible values;
iv coefficient of variation for the measured parameter falls within the range of 0 to 0.3454;
v coefficient of variation for the measured parameter falls within the range of 0.3455 to 0.8;
vi coefficient of variation for the measured parameter is more than 0.8.

This useful feature makes it possible to use the data table consisting of only ones and zeroes, which makes it easier and quicker to process by a computer.

A version of the invention includes principal component analysis as statistical method to reduce the dimension of attribute space and retain as much useful information as possible.

A version of the invention includes cluster analysis as static method. This useful feature makes it possible to divide the plurality of objects and attributes under research into homogenous, to some extent, groups or clusters. This statistical method is multivariate, therefore, it is implied that the source data can have a great volume, i.e. both the number of objects under research and the number of attributes of these objects can be significant.

A version of the invention includes factor analysis as static method. This useful feature makes it possible to analyze the effect of individual factors on a performance indicator using deterministic or stochastic approaches.

A version of the invention includes nonlinear multiple regression as static method.

This useful feature makes it possible to simulate experimental data with the function which is a nonlinear combination of model parameters dependent on one or more independent variables. The data are approximated by sequential approximations.

The combination of the essential features of the claimed invention is not known from the prior art for similar methods, therefore the invention possesses the required feature of novelty pertaining to the method. The proposed task is important and still unresolved, therefore, the invention possesses the required feature of inventive step.

### BRIEF DESCRIPTION OF DRAWINGS

Other distinguishing features and advantages of the invention are readily apparent from the description below which includes but is not limited to the following features, with reference to the figures attached:

FIG. 1 is the system for generating individual nutritional recommendations for a user, according to the invention;

FIG. 2 are the stages of the method for generating individual nutritional recommendations for a user, according to the invention;

FIG. 3 is a diagram of correlations of product and user parameters, according to the invention.

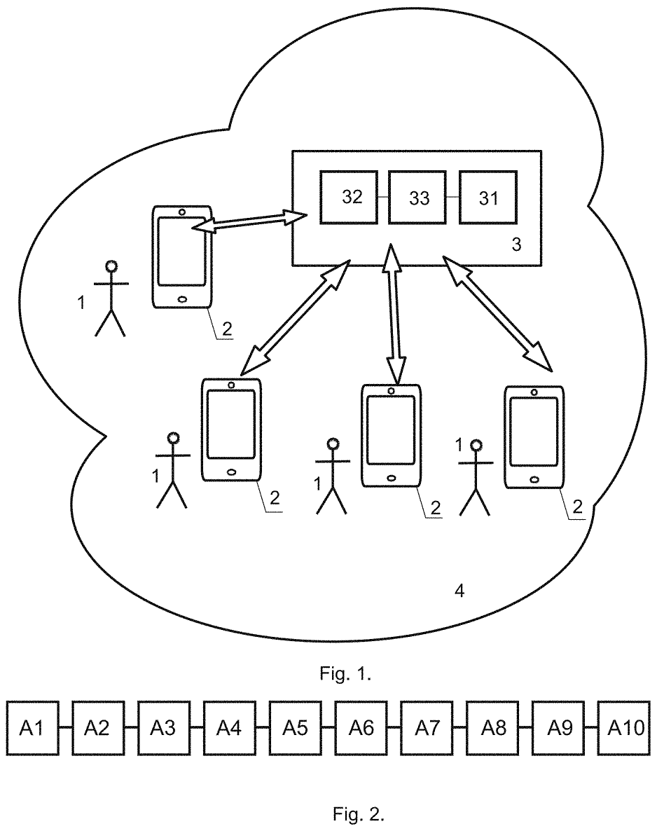

According to FIG. 1, a plurality of users 1 and their personal computer devices 2 (typical examples: laptop, smartphone) connect to a remote server 3 via a unified network 4 which includes any wireless connections (a typical example is Internet). The server 3 hosts a food product database stored in the database storage module 31.
Users transfer their parameters to the servers to be recorded in the user parameter database stored in the database storage module 32.

Via processor 33 connected to modules 31 and 32, a personal matrix of each product effect on user parameters is generated automatically, and the data are returned to the user 1 computer devices 2.

Machine learning and neural network methods can be used for process automation.

### EMBODIMENT OF THE INVENTION

The method for generating individual nutritional recommendations for a user functions as follows (A non-limiting example of embodiment according to the FIG. 1 is provided).
Phase A1. Parameters characterizing the user's condition are measured including complete blood count, pathologies are identified, and individual user data is converted into a numerical sequence. These parameters are introduced into the user personal computer devices 2.
Phase A2. Each product is assigned a corresponding numerical sequence depending on the individual nutrient levels. These data are recorded in the database stored in the database storage module 31.
Phase A3. Correlations between individual user parameters and food parameters are calculated; for this purpose a processor 33 is used to build a personal matrix of each product effect on the user parameters, and factorial weights of each individual product effect on each user parameter are calculated.
Phase A4. Factorial weights of each individual product effect on the user pathology are calculated.
Phase A5. The personal matrix is built as follows: the matrix cells are filled with values (these values can optionally be 0 and 1, corresponding to “no” and “yes”) for each measured parameter divided into six ranges, namely:

i the value of the measured parameter falls within the range of permissible values for said parameter;

ii the value of the measured parameter is more than the average value of the range of permissible values;

iii the value of the measured parameter is less than the average value of the range of permissible values;

iv coefficient of variation for the measured parameter falls within the range of 0 to 0.3454;

v coefficient of variation for the measured parameter falls within the range of 0.3455 to 0.8;

vi coefficient of variation for the measured parameter is more than 0.8.
Phase A6. Principal component analysis is selected as statistical method.
Phase A7. Cluster analysis is used as static method.
Phase A8. Factor analysis is used as static method.
Phase A9. Nonlinear multiple regression is selected as static method.
Phase A10. The resulting personal matrix of each product effect on user parameters is statistically processed using the processor; the matrix is further employed to generate recommendations for the user, wherein products with negative factorial weights are not recommended and products with positive factorial weights are recommended.

### INDUSTRIAL APPLICABILITY

The claimed method for generating individual nutritional recommendations for a user may be implemented by a person skilled in the art in practice and ensures that the claimed objectives are met after implementation, which leads to the conclusion that the invention meets the requirement of “industrial applicability”.

The claimed method for generating individual nutritional recommendations for a user is based on conventional technology and can be embodied without any additional technical challenges.

### Examples

Method testing involved randomized sampling of 17 food products from 10 000 food products, two blood test results from two individual patients, two pathology descriptions from the original medical cases of the above two individual patients; these data were assigned to the horizontal cells of the matrix. (see Table 1).

TABLE 1
List of randomly sampled food products

| No | Food product or user parameter |
|---|---|
| 1 | Wheat flour |
| 2 | Squash |
| 3 | Red carrot |
| 4 | Red bell pepper |
| 5 | Horseradish |
| 6 | Sorrel |
| 7 | Rice |
| 8 | Pork sirloin |
| 9 | Tuna |
| 10 | Bananas |
| 11 | Lemon |
| 12 | Bilberry |
| 13 | Condensed milk |
| 14 | Half-and-half cottage cheese |
| 15 | 6% fat baked yogurt |
| 16 | Yeast |
| 17 | Hazelnut |
| 18 | Blood test from Patient 1 |
| 19 | Atherosclerosis |
| 20 | Blood test from Patient 2 |
| 21 | Osteoporosis |

Following that, the food and user parameters were selected. The scope of parameters included 52 organic and inorganic components in the food products, blood test results, and pathology description. Each component and parameter was statistically described in absolute numbers. Any component was represented in six ranges:
i the value of the measured parameter falls within the range of permissible values for said parameter;
ii the value of the measured parameter is more than the average value of the range of permissible values;
iii the value of the measured parameter is less than the average value of the range of permissible values;
iv coefficient of variation for the measured parameter falls within the range of 0 to 0.34;
v coefficient of variation for the measured parameter falls within the range of 0.3455 to 0.8;
vi coefficient of variation for the measured parameter is more than 0.8.

All matrix cells in the columns describe the above 52 parameters using 312 indicators (see Table 2), wherein parameters relate to both product and user parameters.

TABLE 2
List of parameters under research for selected food products and user parameters.

| No | Parameter | i | ii | iii | iv | v | vi |
|---|---|---|---|---|---|---|---|
| 1 | Protein | X | >X | <X | 0.0-34.54 | 34.55-80.0 | >80.0 |
| 2 | Fat | X | >X | <X | 0.0-34.54 | 34.55-80.0 | >80.0 |
| 3 | Carbohydrates | X | >X | <X | 0.0-34.54 | 34.55-80.0 | >80.0 |
| 4 | Potassium | X | >X | <X | 0.0-34.54 | 34.55-80.0 | >80.0 |
| 5 | Calcium | X | >X | <X | 0.0-34.54 | 34.55-80.0 | >80.0 |
| 6 | Magnesium | X | >X | <X | 0.0-34.54 | 34.55-80.0 | >80.0 |
| 7 | Sodium | X | >X | <X | 0.0-34.54 | 34.55-80.0 | >80.0 |
| 8 | Phosphorus | X | >X | <X | 0.0-34.54 | 34.55-80.0 | >80.0 |
| 9 | Iron | X | >X | <X | 0.0-34.54 | 34.55-80.0 | >80.0 |
| 10 | Iodine | X | >X | <X | 0.0-34.54 | 34.55-80.0 | >80.0 |
| 11 | Cobalt | X | >X | <X | 0.0-34.54 | 34.55-80.0 | >80.0 |
| 12 | Manganese | X | >X | <X | 0.0-34.54 | 34.55-80.0 | >80.0 |
| 13 | Copper | X | >X | <X | 0.0-34.54 | 34.55-80.0 | >80.0 |
| 14 | Molybdenum | X | >X | <X | 0.0-34.54 | 34.55-80.0 | >80.0 |
| 15 | Fluorine | X | >X | <X | 0.0-34.54 | 34.55-80.0 | >80.0 |
| 16 | Zinc | X | >X | <X | 0.0-34.54 | 34.55-80.0 | >80.0 |
| 17 | A-retinol | X | >X | <X | 0.0-34.54 | 34.55-80.0 | >80.0 |
| 18 | B-carotene | X | >X | <X | 0.0-34.54 | 34.55-80.0 | >80.0 |
| 19 | E-tocopherol | X | >X | <X | 0.0-34.54 | 34.55-80.0 | >80.0 |
| 20 | C-ascorbic acid | X | >X | <X | 0.0-34.54 | 34.55-80.0 | >80.0 |
| 21 | Vitamin B1 | X | >X | <X | 0.0-34.54 | 34.55-80.0 | >80.0 |
| 22 | Vitamin B2 | X | >X | <X | 0.0-34.54 | 34.55-80.0 | >80.0 |
| 23 | Folic acid | X | >X | <X | 0.0-34.54 | 34.55-80.0 | >80.0 |
| 24 | PP-niacin | X | >X | <X | 0.0-34.54 | 34.55-80.0 | >80.0 |
| 25 | Energy value | X | >X | <X | 0.0-34.54 | 34.55-80.0 | >80.0 |
| 26 | Acid-alkaline balance | X | >X | <X | 0.0-34.54 | 34.55-80.0 | >80.0 |
| 27 | White blood cells | X | >X | <X | 0.0-34.54 | 34.55-80.0 | >80.0 |
| 28 | Red blood cells | X | >X | <X | 0.0-34.54 | 34.55-80.0 | >80.0 |
| 29 | Hemoglobin | X | >X | <X | 0.0-34.54 | 34.55-80.0 | >80.0 |
| 30 | Platelets | X | >X | <X | 0.0-34.54 | 34.55-80.0 | >80.0 |
| 31 | Bilirubin | X | >X | <X | 0.0-34.54 | 34.55-80.0 | >80.0 |
| 32 | ALT | X | >X | <X | 0.0-34.54 | 34.55-80.0 | >80.0 |
| 33 | AST | X | >X | <X | 0.0-34.54 | 34.55-80.0 | >80.0 |
| 34 | Cholesterol | X | >X | <X | 0.0-34.54 | 34.55-80.0 | >80.0 |
| 35 | Triglycerides | X | >X | <X | 0.0-34.54 | 34.55-80.0 | >80.0 |
| 36 | Glucose | X | >X | <X | 0.0-34.54 | 34.55-80.0 | >80.0 |
| 37 | Urea | X | >X | <X | 0.0-34.54 | 34.55-80.0 | >80.0 |
| 38 | Creatinine | X | >X | <X | 0.0-34.54 | 34.55-80.0 | >80.0 |
| 39 | S-amylase | X | >X | <X | 0.0-34.54 | 34.55-80.0 | >80.0 |
| 40 | S-peptide | X | >X | <X | 0.0-34.54 | 34.55-80.0 | >80.0 |
| 41 | Ferritin | X | >X | <X | 0.0-34.54 | 34.55-80.0 | >80.0 |
| 42 | Fibrinogen | X | >X | <X | 0.0-34.54 | 34.55-80.0 | >80.0 |
| 43 | LDH | X | >X | <X | 0.0-34.54 | 34.55-80.0 | >80.0 |
| 44 | Chlorine | X | >X | <X | 0.0-34.54 | 34.55-80.0 | >80.0 |
| 45 | D-dimer | X | >X | <X | 0.0-34.54 | 34.55-80.0 | >80.0 |
| 46 | TTH | X | >X | <X | 0.0-34.54 | 34.55-80.0 | >80.0 |
| 47 | PSA | X | >X | <X | 0.0-34.54 | 34.55-80.0 | >80.0 |
| 48 | Monocytes, abs | X | >X | <X | 0.0-34.54 | 34.55-80.0 | >80.0 |
| 49 | IgA | X | >X | <X | 0.0-34.54 | 34.55-80.0 | >80.0 |
| 50 | IgE | X | >X | <X | 0.0-34.54 | 34.55-80.0 | >80.0 |
| 51 | Atherosclerosis | X | >X | <X | 0.0-34.54 | 34.55-80.0 | >80.0 |
| 52 | Osteoporosis | X | >X | <X | 0.0-34.54 | 34.55-80.0 | >80.0 |

When calculated values are added to the table, it looks as follows (the matrix is replaced by a sequence of continued values corresponding to the linear reading of matrix values in the Table 2, for ease of processing):

TABLE 3
Factorial weights of nutrients, blood test values,
pathology parameters.

| | | | Numerical |
|---|---|---|---|
| No | Parameter | i-vi range | value |
| 1 | Proteins | X | 0.00 |
| 2 | Proteins | >X | 0.18 |
| 3 | Proteins | <X | 0.49 |
| 4 | Proteins | 0.0-34.54 | 0.21 |
| 5 | Proteins | 34.55-80.0 | 0.48 |
| 6 | Proteins | >80.00 | 0.66 |
| 7 | Fats | X | 0.15 |
| 8 | Fats | >X | 0.11 |
| 9 | Fats | <X | 0.55 |
| 10 | Fats | 0.0-34.54 | 0.04 |
| 11 | Fats | 34.55-80.0 | 0.47 |
| 12 | Fats | >80.00 | 0.17 |
| 13 | Carbohydrates | X | 0.12 |
| 14 | Carbohydrates | >X | −0.22 |
| 15 | Carbohydrates | <X | 0.37 |
| 16 | Carbohydrates | 0.0-34.54 | 0.17 |
| 17 | Carbohydrates | 34.55-80.0 | 0.42 |
| 18 | Carbohydrates | >80.00 | 0.12 |
| 19 | Potassium | X | 0.66 |
| 20 | Potassium | >X | 0.14 |
| 21 | Potassium | <X | −0.40 |
| 22 | Potassium | 0.0-34.54 | −0.39 |
| 23 | Potassium | 34.55-80.0 | 0.45 |
| 24 | Potassium | >80.00 | 0.05 |
| 25 | Calcium | X | 0.10 |
| 26 | Calcium | >X | −0.50 |
| 27 | Calcium | <X | 0.44 |
| 28 | Calcium | 0.0-34.54 | −0.55 |
| 29 | Calcium | 34.55-80.0 | −0.56 |
| 30 | Calcium | >80.00 | 0.10 |
| 31 | Magnesium | X | 0.01 |
| 32 | Magnesium | >X | 0.16 |
| 33 | Magnesium | <X | 0.15 |
| 34 | Magnesium | 0.0-34.54 | −0.57 |
| 35 | Magnesium | 34.55-80.0 | 0.51 |
| 36 | Magnesium | >80.00 | −0.10 |
| 37 | Sodium | X | 0.10 |
| 38 | Sodium | >X | −0.76 |
| 39 | Sodium | <X | 0.16 |
| 40 | Sodium | 0.0-34.54 | 0.80 |
| 41 | Sodium | 34.55-80.0 | 0.57 |
| 42 | Sodium | >80.00 | 0.15 |
| 43 | Phosphorus | X | 0.10 |
| 44 | Phosphorus | >X | 0.15 |
| 45 | Phosphorus | <X | −0.09 |
| 46 | Phosphorus | 0.0-34.54 | −0.61 |
| 47 | Phosphorus | 34.55-80.0 | 0.54 |
| 48 | Phosphorus | >80.00 | 0.10 |
| 49 | Iron | X | 0.10 |
| 50 | Iron | >X | 0.14 |
| 51 | Iron | <X | 0.51 |
| 52 | Iron | 0.0-34.54 | 0.10 |
| 53 | Iron | 34.55-80.0 | 0.49 |
| 54 | Iron | >80.00 | 0.67 |
| 55 | Iodine | X | 0.66 |
| 56 | Iodine | >X | −0.13 |
| 57 | Iodine | <X | −0.61 |
| 58 | Iodine | 0.0-34.54 | −0.10 |
| 59 | Iodine | 34.55-80.0 | −0.84 |
| 60 | Iodine | >80.00 | −0.36 |
| 61 | Cobalt | X | −0.05 |
| 62 | Cobalt | >X | −0.05 |
| 63 | Cobalt | <X | −0.67 |
| 64 | Cobalt | 0.0-34.54 | −0.10 |
| 65 | Cobalt | 34.55-80.0 | −0.02 |
| 66 | Cobalt | >80.00 | −0.40 |
| 67 | Manganese | X | −0.01 |
| 68 | Manganese | >X | −0.05 |
| 69 | Manganese | <X | −0.63 |
| 70 | Manganese | 0.0-34.54 | −0.10 |
| 71 | Manganese | 34.55-80.0 | −0.29 |
| 72 | Manganese | >80.00 | −0.33 |
| 73 | Copper | X | −0.05 |
| 74 | Copper | >X | −0.14 |
| 75 | Copper | <X | −0.06 |
| 76 | Copper | 0.0-34.54 | 0.81 |
| 77 | Copper | 34.55-80.0 | −0.33 |
| 78 | Copper | >80.00 | −0.32 |
| 79 | Molybdenum | X | −0.05 |
| 80 | Molybdenum | >X | −0.11 |
| 81 | Molybdenum | <X | −0.54 |
| 82 | Molybdenum | 0.0-34.54 | −0.10 |
| 83 | Molybdenum | 34.55-80.0 | −0.19 |
| 84 | Molybdenum | >80.00 | −0.38 |
| 85 | Fluorine | X | −0.10 |
| 86 | Fluorine | >X | −0.15 |
| 87 | Fluorine | <X | −0.45 |
| 88 | Fluorine | 0.0-34.54 | −0.10 |
| 89 | Fluorine | 34.55-80.0 | −0.22 |
| 90 | Fluorine | >80.00 | −0.34 |
| 91 | Zinc | X | −0.10 |
| 92 | Zinc | >X | 0.01 |
| 93 | Zinc | <X | 0.10 |
| 94 | Zinc | 0.0-34.54 | −0.81 |
| 95 | Zinc | 34.55-80.0 | 0.34 |
| 96 | Zinc | >80.00 | 0.31 |
| 97 | A-retinol | X | 0.30 |
| 98 | A-retinol | >X | 0.11 |
| 99 | A-retinol | <X | 0.34 |
| 100 | A-retinol | 0.0-34.54 | 0.10 |
| 101 | A-retinol | 34.55-80.0 | 0.20 |
| 102 | A-retinol | >80.00 | 0.25 |
| 103 | B-carotene | X | 0.01 |
| 104 | B-carotene | >X | 0.10 |
| 105 | B-carotene | <X | 0.64 |
| 106 | B-carotene | 0.0-34.54 | 0.03 |
| 107 | B-carotene | 34.55-80.0 | 0.39 |
| 108 | B-carotene | >80.00 | −0.28 |
| 109 | E-tocopherol | X | 0.72 |
| 110 | E-tocopherol | >X | 0.29 |
| 111 | E-tocopherol | <X | 0.36 |
| 112 | E-tocopherol | 0.0-34.54 | 0.10 |
| 113 | E-tocopherol | 34.55-80.0 | 0.26 |
| 114 | E-tocopherol | >80.00 | 0.35 |
| 151 | C-ascorbic acid | X | 0.01 |
| 116 | C-ascorbic acid | >X | 0.11 |
| 117 | C-ascorbic acid | <X | 0.64 |
| 118 | C-ascorbic acid | 0.0-34.54 | −0.38 |
| 119 | C-ascorbic acid | 34.55-80.0 | 0.48 |
| 120 | C-ascorbic acid | >80.00 | 0.17 |
| 121 | Vitamin B1 | X | 0.14 |
| 122 | Vitamin B1 | >X | 0.11 |
| 123 | Vitamin B1 | <X | 0.49 |
| 124 | Vitamin B1 | 0.0-34.54 | 0.1 |
| 125 | Vitamin B1 | 34.55-80.0 | 0.62 |
| 126 | Vitamin B1 | >80.00 | 0.72 |
| 127 | Vitamin B2 | X | 0.61 |
| 128 | Vitamin B2 | >X | −0.15 |
| 129 | Vitamin B2 | <X | 0.41 |
| 130 | Vitamin B2 | 0.0-34.54 | 0.16 |
| 131 | Vitamin B2 | 34.55-80.0 | −0.41 |
| 132 | Vitamin B2 | >80.00 | 0.03 |
| 133 | Folic acid | X | 0.02 |
| 134 | Folic acid | >X | 0.10 |
| 135 | Folic acid | <X | 0.63 |
| 136 | Folic acid | 0.0-34.54 | 0.10 |
| 137 | Folic acid | 34.55-80.0 | 0.33 |
| 138 | Folic acid | >80.00 | 0.29 |
| 139 | PP-niacin | X | 0.16 |
| 140 | PP-niacin | >X | −0.14 |
| 141 | PP-niacin | <X | 0.42 |
| 142 | PP-niacin | 0.0-34.54 | 0.1 |
| 143 | PP-niacin | 34.55-80.0 | 0.59 |
| 144 | PP-niacin | >80.00 | −0.63 |
| 145 | Energy value | X | 0.02 |
| 146 | Energy value | >X | 0.18 |
| 147 | Energy value | <X | −0.26 |
| 148 | Energy value | 0.0-34.54 | 0.20 |
| 149 | Energy value | 34.55-80.0 | −0.36 |
| 150 | Energy value | >80.00 | 0.72 |
| 151 | Acid-alkaline balance | X | −0.29 |
| 152 | Acid-alkaline balance | >X | −0.22 |
| 153 | Acid-alkaline balance | <X | −0.16 |
| 154 | Acid-alkaline balance | 0.0-34.54 | −0.10 |
| 155 | Acid-alkaline balance | 34.55-80.0 | −0.24 |
| 156 | Acid-alkaline balance | >80.00 | −0.34 |
| 157 | White blood cells | X | 0.02 |
| 158 | White blood cells | >X | 0.64 |
| 159 | White blood cells | <X | −0.77 |
| 160 | White blood cells | 0.0-34.54 | −0.97 |
| 161 | White blood cells | 34.55-80.0 | 0.02 |
| 162 | White blood cells | >80.00 | 0.03 |
| 163 | Red blood cells | X | −0.10 |
| 164 | Red blood cells | >X | −0.70 |
| 165 | Red blood cells | <X | 0.64 |
| 166 | Red blood cells | 0.0-34.54 | −0.97 |
| 167 | Red blood cells | 34.55-80.0 | −0.10 |
| 168 | Red blood cells | >80.00 | 0.03 |
| 169 | Hemoglobin | X | 0.02 |
| 170 | Hemoglobin | >X | 0.03 |
| 171 | Hemoglobin | <X | 0.98 |
| 172 | Hemoglobin | 0.0-34.54 | −0.97 |
| 173 | Hemoglobin | 34.55-80.0 | −0.09 |
| 174 | Hemoglobin | >80.00 | −0.97 |
| 175 | Platelets | X | −0.10 |
| 176 | Platelets | >X | −0.63 |
| 177 | Platelets | <X | −0.70 |
| 178 | Platelets | 0.0-34.54 | 0.99 |
| 179 | Platelets | 34.55-80.0 | −0.10 |
| 180 | Platelets | >80.00 | −0.05 |
| 181 | Bilirubin | X | −0.10 |
| 182 | Bilirubin | >X | −0.97 |
| 183 | Bilirubin | <X | −0.70 |
| 184 | Bilirubin | 0.0-34.54 | −0.63 |
| 185 | Bilirubin | 34.55-80.0 | −0.10 |
| 186 | Bilirubin | >80.00 | 0.03 |
| 187 | ALT | X | 0.10 |
| 188 | ALT | >X | −0.97 |
| 189 | ALT | <X | 0.72 |
| 190 | ALT | 0.0-34.54 | 0.02 |
| 191 | ALT | 34.55-80.0 | −0.53 |
| 192 | ALT | >80.00 | 0.10 |
| 193 | AST | X | 0.11 |
| 194 | AST | >X | −0.10 |
| 195 | AST | <X | −0.97 |
| 196 | AST | 0.0-34.54 | −0.10 |
| 197 | AST | 34.55-80.0 | −0.97 |
| 198 | AST | >80.00 | −0.10 |
| 199 | Cholesterol | X | −0.10 |
| 200 | Cholesterol | >X | −0.97 |
| 201 | Cholesterol | <X | −0.10 |
| 202 | Cholesterol | 0.0-34.54 | −0.97 |
| 203 | Cholesterol | 34.55-80.0 | −0.10 |
| 204 | Cholesterol | >80.00 | 0.03 |
| 205 | Triglycerides | X | −0.10 |
| 206 | Triglycerides | >X | 0.02 |
| 207 | Triglycerides | <X | −0.97 |
| 208 | Triglycerides | 0.0-34.54 | −0.97 |
| 209 | Triglycerides | 34.55-80.0 | −0.10 |
| 210 | Triglycerides | >80.00 | −0.10 |
| 211 | Glucose | X | 0.03 |
| 212 | Glucose | >X | 0.99 |
| 213 | Glucose | <X | 0.02 |
| 214 | Glucose | 0.0-34.54 | 0.99 |
| 215 | Glucose | 34.55-80.0 | −0.10 |
| 216 | Glucose | >80.00 | −0.10 |
| 217 | Urea | X | 0.64 |
| 218 | Urea | >X | −0.70 |
| 219 | Urea | <X | −0.71 |
| 220 | Urea | 0.0-34.54 | 0.64 |
| 221 | Urea | 34.55-80.0 | −0.10 |
| 222 | Urea | >80.00 | −0.10 |
| 223 | Creatinine | X | −0.10 |
| 224 | Creatinine | >X | −0.10 |
| 225 | Creatinine | <X | −0.97 |
| 226 | Creatinine | 0.0-34.54 | −0.97 |
| 227 | Creatinine | 34.55-80.0 | 0.03 |
| 228 | Creatinine | >80.00 | −0.10 |
| 229 | S-amylase | X | −0.10 |
| 230 | S-amylase | >X | 0.64 |
| 231 | S-amylase | <X | 0.72 |
| 232 | S-amylase | 0.0-34.54 | −0.10 |
| 233 | S-amylase | 34.55-80.0 | 0.99 |
| 234 | S-amylase | >80.00 | −0.10 |
| 325 | S-peptide | X | −0.10 |
| 236 | S-peptide | >X | −0.10 |
| 237 | S-peptide | <X | 0.85 |
| 238 | S-peptide | 0.0-34.54 | −0.10 |
| 239 | S-peptide | 34.55-80.0 | 0.63 |
| 240 | S-peptide | >80.00 | −0.50 |
| 241 | Ferritin | X | −0.10 |
| 242 | Ferritin | >X | −0.10 |
| 243 | Ferritin | <X | −0.97 |
| 244 | Ferritin | 0.0-34.54 | −0.10 |
| 245 | Ferritin | 34.55-80.0 | −0.97 |
| 246 | Ferritin | >80.00 | 0.20 |
| 247 | Fibrinogen | X | −0.10 |
| 428 | Fibrinogen | >X | −0.70 |
| 249 | Fibrinogen | <X | −0.61 |
| 250 | Fibrinogen | 0.0-34.54 | −0.97 |
| 251 | Fibrinogen | 34.55-80.0 | −0.10 |
| 252 | Fibrinogen | >80.00 | −0.10 |
| 253 | LDH | X | −0.10 |
| 254 | LDH | >X | 0.03 |
| 255 | LDH | <X | 0.99 |
| 256 | LDH | 0.0-34.54 | 0.99 |
| 257 | LDH | 34.55-80.0 | −0.10 |
| 258 | LDH | >80.00 | −0.10 |
| 259 | Chlorine | X | −0.10 |
| 260 | Chlorine | >X | 0.99 |
| 261 | Chlorine | <X | −0.10 |
| 262 | Chlorine | 0.0-34.54 | 0.99 |
| 263 | Chlorine | 34.55-80.0 | −0.10 |
| 264 | Chlorine | >80.00 | −0.10 |
| 265 | D-dimer | X | −0.10 |
| 266 | D-dimer | >X | 0.99 |
| 267 | D-dimer | <X | −0.10 |
| 268 | D-dimer | 0.0-34.54 | 0.03 |
| 269 | D-dimer | 34.55-80.0 | 0.49 |
| 270 | D-dimer | >80.00 | −0.10 |
| 271 | TTH | X | −0.10 |
| 272 | TTH | >X | 0.99 |
| 273 | TTH | <X | 0.99 |
| 274 | TTH | 0.0-34.54 | −0.10 |
| 275 | TTH | 34.55-80.0 | 0.99 |
| 276 | TTH | >80.00 | −0.10 |
| 277 | PSA | X | −0.10 |
| 278 | PSA | >X | 0.03 |
| 279 | PSA | <X | 0.64 |
| 280 | PSA | 0.0-34.54 | −0.10 |
| 281 | PSA | 34.55-80.0 | −0.10 |
| 282 | PSA | >80.00 | −0.97 |
| 283 | Monocytes abs | X | −0.89 |
| 284 | Monocytes abs | >X | 0.64 |
| 285 | Monocytes abs | <X | −0.70 |
| 286 | Monocytes abs | 0.0-34.54 | 0.72 |
| 287 | Monocytes abs | 34.55-80.0 | 0.03 |
| 288 | Monocytes abs | >80.00 | 0.72 |
| 289 | IgA | X | 0.01 |
| 290 | IgA | >X | 0.72 |
| 291 | IgA | <X | 0.99 |
| 292 | IgA | 0.0-34.54 | 0.72 |
| 293 | IgA | 34.55-80.0 | 0.64 |
| 294 | IgA | >80.00 | −0.10 |
| 295 | IgE | X | −0.10 |
| 296 | IgE | >X | −0.70 |
| 297 | IgE | <X | 0.66 |
| 298 | IgE | 0.0-34.54 | 0.96 |
| 299 | IgE | 34.55-80.0 | 0.03 |
| 300 | IgE | >80.00 | −0.10 |
| 301 | Atherosclerosis | X | −0.10 |
| 302 | Atherosclerosis | >X | −0.86 |
| 303 | Atherosclerosis | <X | −0.10 |
| 304 | Atherosclerosis | 0.0-34.54 | −0.70 |
| 305 | Atherosclerosis | 34.55-80.0 | −0.52 |
| 306 | Atherosclerosis | >80.00 | −0.10 |
| 307 | Osteoporosis | X | −0.10 |
| 308 | Osteoporosis | >X | −0.70 |
| 309 | Osteoporosis | <X | −0.10 |
| 310 | Osteoporosis | 0.0-34.54 | 0.03 |
| 311 | Osteoporosis | 34.55-80.0 | 0.72 |
| 312 | Osteoporosis | >80.00 | −0.10 |

Following that, principal component analysis, cluster analysis, factorial analysis, and nonlinear multiple regression are applied to process the above nutrients, blood test values, pathology parameters, and obtain the following table:

TABLE 4
Calculated factorial weights of nutrients, blood test values,
pathology parameters.

| No | Product or user parameter | Value |
|---|---|---|
| 1 | Wheat flour | −0.66 |
| 2 | Squash | −0.83 |
| 3 | Red carrot | −0.76 |
| 4 | Red bell pepper | −0.77 |
| 5 | Horseradish | −0.81 |
| 6 | Sorrel | −0.73 |
| 7 | Rice | −0.44 |
| 8 | Pork sirloin | −0.67 |
| 9 | Tuna | −0.76 |
| 10 | Bananas | −0.74 |
| 11 | Lemon | −0.49 |
| 12 | Bilberry | −0.79 |
| 13 | Condensed milk | −0.83 |
| 14 | Half-and-half cottage cheese | −0.84 |
| 15 | 6% fat baked yogurt | −0.53 |
| 16 | Yeast | −0.68 |
| 17 | Hazelnut | −0.63 |
| 18 | Blood test of patient 1 | +0.11 |
| 19 | Atherosclerosis | +0.10 |
| 20 | Blood test of patient 2 | +0.14 |
| 21 | Osteoporosis | +0.10 |

TABLE 5
Ranged rows of absolute and relative values of nonlinear
multiple regression equation coefficients for nutrients,
blood test values, pathology parameters.

| | | Equation | Share of | Share of |
|---|---|---|---|---|
| | | coefficient | positive | negative |
| | | values, | factors, | factors, |
| | Designations | abs | % | % |
| No | 1 | 2 | 3 | 4 |
| 1. | Hazelnut (17) | 2.815 | 66.5 | 33.5 |
| 2. | Pork sirloin (8) | 2.791 | 67.9 | 32.1 |
| 3. | Tuna (9) | 2.570 | 69.6 | 30.4 |
| 4. | Red carrot (3) | 2.567 | 55.7 | 44.3 |
| | Average, Group 1 | 2.686 | 64.9 | 35.1 |
| 5. | Bananas (10) | 2.414 | 66.0 | 34.0 |
| 6. | Yeast (16) | 2.354 | 64.3 | 35.7 |
| 7. | Rice (7) | 2.316 | 73.7 | 26.3 |
| 8. | Red bell pepper (4) | 2.163 | 76.8 | 23.2 |
| 9. | Lemon (11) | 2.100 | 64.8 | 35.2 |
| 10. | Condensed milk (13) | 2.052 | 93.0 | 7.0 |
| 11. | Half-and-half cottage cheese | 2.044 | 80.3 | 19.7 |
| | (14) | | | |
| 12. | Sorrel (6) | 1.976 | 68.5 | 31.5 |
| 13. | Horseradish (5) | 1.971 | 78.6 | 21.4 |
| 14. | Bilberry (12) | 1.887 | 80.6 | 19.4 |
| 15. | Squash (2) | 1.763 | 77.3 | 22.7 |
| 16. | Blood test of patient 2 | 1.762 | 66.5 | 35.5 |
| | (20) | | | |
| 17. | Wheat flour (1) | 1.635 | 90.2 | 9.8 |
| | Average, Group 2 | 2.034 | 75.9 | 24.1 |
| 18. | 6% baked yogurt (15) | 1.179 | 75.5 | 24.5 |
| 19. | Blood test of patient 1 (18) | 0.888 | 72.6 | 27.4 |
| 20. | Osteoporosis (21) | 0.773 | 53.4 | 46.6 |
| 21. | Atherosclerosis | 0.243 | 48.2 | 52.8 |
| | Average, Group 3 | 0.771 | 62.2 | 37.8 |

Note:
Column (2) lists the sum of independent variables as the value of dependent variable.

Columns (3) and (4) specify relative positive or negative sums of independent variables that have a corresponding effect to the dependent variable specified in column (1).

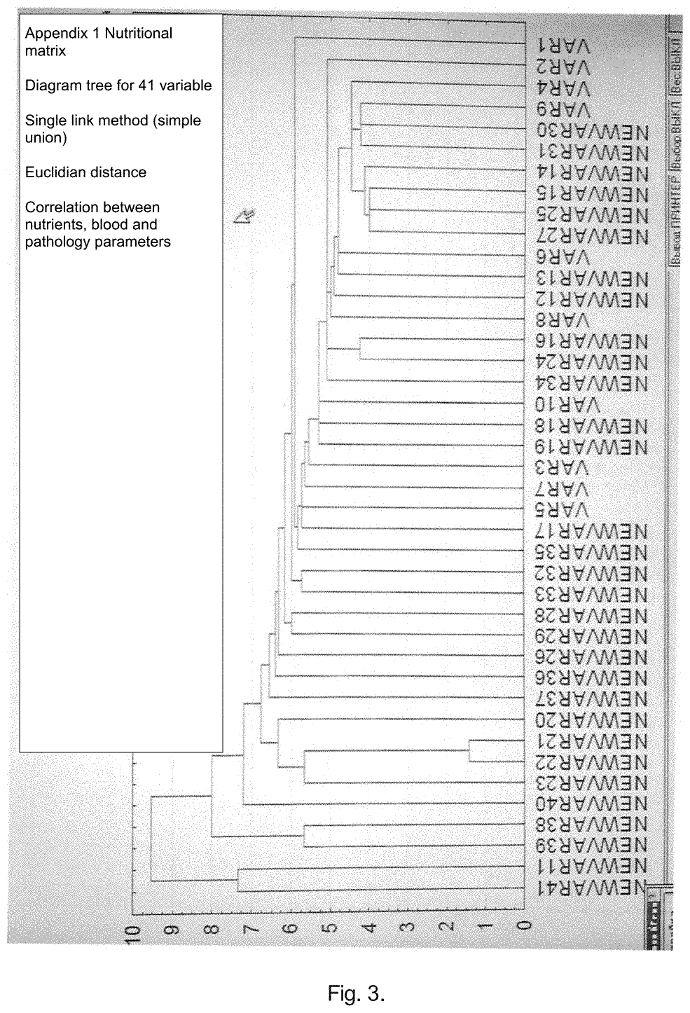

As is evident from FIG. 3, the products from the randomly sampled set of 17 products have specific structural and correlating relations with the blood tests values of the two patients and their pathologies at the start of digestive process. 41 parameter of the studied 52 parameters have an effect non equal to zero.

The system of correlations between food products, blood test and pathology parameters, as we can see from the FIG. 3, results in seven interrelated structures.

It is noteworthy that blood test and pathology parameters from the two patients formed an integrated structure with low but positive factorial weights of confidence factor.

Therefore, the calculations show that health status is determined by inorganic and organic nutrients present in the food products, that results from precise measurements and not dietician fantasy.

It must be noted that only two food product parameters (Rice—number 7 in Table 1 and 6% fat baked yogurt—number 15 in the Table 1) were close to the blood test and pathology values and interconnected with the same.

The measured attributes of food products directly related to blood test and pathology parameters have negative factorial weights. It means that these products in this set have negative values and negative effect on both blood test parameters and their pathologies, or the effect of these two food products is essentially linear.

All other food products were only indirectly related to the blood test and pathology parameters. It is important that all other food products formed correlations with other food products and that absolutely all these correlations have high but negative factorial weights. It shows that the quality of the randomly sampled product set was highly uneven and had different effect on the quality of life.

This problem was solved by nonlinear multiple regression as a statistical method wherein all food products with their organic and inorganic components were in turns taken as dependent and independent variables. The result was the table with ranged rows of equation coefficient values for both dependent and independent variables (see Table 5).

The ranged data of nonlinear multiple regression equation coefficients for dependent variables clearly show that all food products, blood test values and pathologies break down into three groups.

Group 1 has the highest activity of organic and inorganic product, blood test, and pathology parameters, judging from the sums of regression equation coefficients for dependent variables. A notable feature of this group of organic and inorganic nutrients is that they demonstrate a high level of correlations with other system elements (see FIG. 3) compared to other groups.

It should be noted that less than two thirds of the list of biomaterials from the Table 5 has a positive effect on each dependent variable and that over a third or nutrients has a negative effect on each dependent variable listed in column 2 of the Table 5. In other words, the effect of nutrients, blood test and pathology parameters on each other is nonlinear, as was proved by measurements.

The above conclusion can be verified by the analysis of Group 2 of ranged nutrient coefficients that demonstrates lower activity of the same as dependent variables. It is noteworthy that the lower the activity of organic and inorganic nutrients, the higher the share of independent variables from the list of products with a positive effect on a dependent variable as a product, or over two thirds. The share of independent variables (as food products) with a negative effect on each other is reduced respectively.

The study of Group 3 of ranged biomaterial coefficients that includes mostly blood test values from two patients and pathology parameters confirms, on the one hand, a higher positive effect of independent variables as food products on dependent variables (blood tests and pathologies). On the other hand, the share of negative effect of food products on blood test and pathology parameters is relatively smaller.

At the same time, interesting observations can be derived from the nonlinear nature of correlations between food products and blood test and pathology parameters.

It is evident that randomly sampled food products as dependent variables, though nonlinear, have a minimum effect on osteoporosis (+53.4 and −46.6). At the same time, the same set of products has a negative effect or helps decrease atherosclerosis in the other patient (+48.2% and −52.8% respectively, see Table 1).

Therefore, two fundamental factors have to be taken into account when choosing food products: firstly, the factor of nonlinear correlations between food products, secondly, the probability of competing effect of personal tastes on individual pathologies and/or a plurality of pathologies.

The table of factorial weights (see Table 4) of organic and inorganic nutrients, blood and pathology parameters confirms both the nonlinearity of the system of correlations and competing effect on each other.

At least two observations are important in this system of correlations between organic and inorganic nutrients, blood and pathology parameters that falls into seven macrostructures. The first observation is that all structures of correlations between nutrients and blood and pathology parameters are, in a sense, “suspended” on six close interconnections of parameters (from A-retinol to Vitamin B) and zinc (a heavy metal). The second observation is that all blood parameters have strong negative interconnections, judging from factorial weights, with proteins, fats and carbohydrates in food products.

In addition, the correlation systems clearly shows that blood parameters are interconnected with fats as nutrients. This correlation once again confirms the fact proven by traditional medical studies, or that the way the nutrients are absorbed in the digestive system depends on their solubility in lipids.

In general, as the results of statistical analysis show, correlations between nutrients and blood and pathology parameters are nonlinear both for food products and for blood and pathology parameters. That is why it is wrong to recommend a food product judging by the highest levels of a nutrient if a patient has a deficiency of said nutrient. Selection of food products for individual patients with pathologies must be based on mass calculations that allow for implementing this invention patent.

The data in the Table 4 aggregate meaningful data and directions for all nutrients and blood parameters as dependent variables. The left to right diagonal of the matrix table splits the aggregated data system into positive and negative data groups.

Above the diagonal are positive values of the sums of dependent variables of food products and blood and pathology parameters. Negative values of the sums of dependent variables of food products and blood and pathology parameters are located below the diagonal.

It is noteworthy that all measurement data that are at least three percentage points above the threshold are meaningful. The subject of analysis will be cross-sections of correlations numbering at least three correlations. A system of cross-sections of correlations will be called “cores” for ease of naming.

The first thing that is observed is that positive cores above the diagonal are three times as many as the negative cores below the diagonal. In other words, the set of products selected for analysis largely concentrates positive aggregated total effect.

An interesting fact is that the same core of correlations between the nutrients can have a positive or a negative effect. Let's look at the example. A positive core comprises correlations of the following parameters: wheat flour (number 1 in the Table 1), squash (number 2 in the Table 1), red carrot (number 3 in the Table 1), yeast (number 16 in the Table 1), hazelnut (number 17 in the Table 1), the average correlation value being 6.82%. The negative core comprises negative correlations for the same products with the average correlation value of 6.2%. That is, there are more positive correlation cores than negative correlation cores in numerical terms. In general, positive correlation cores are not only more numerous, but also much stronger.

Besides, the system of matrix correlation demonstrates that statistical methods can be useful tools for research and measurement of nutrient effect on blood and pathology parameters, excluding the nonsense proposed by dieticians.

The demonstrated matrix table (see Table 4) of correlations between nutrients and blood and pathology parameters has one positive correlation core: blood test of patient 1 (number 18 in the Table 1), atherosclerosis (number 19 in the Table 1), blood test of patient 2 (number 20 in the Table 1), osteoporosis (number 21 in the Table 1). This system of correlations in the nutritional paradigm demonstrates only the fact that the blood test values and pathologies are integrated in a dedicated nutritional cluster. During the further correlation analysis, it becomes evident that nutrients of the selected set have practically no positive effect on blood test of patient 1 (number 18 in the Table 1). The only product that has a negative effect on blood test of patient 1 is sorrel (number 6 in the Table 1). As for atherosclerosis (number 19 in the Table 1), the matrix table shows that two parameters have a marked positive effect on this pathology, firstly, squash (number 2 in the Table 1) and blood test of patient 1.

The analysis of blood test of patient 2 in the nutrient paradigm is quite different. On average, the nutrients of the following products have a positive effect on blood test of patient 2 with the weight of 19.26%: horseradish (number 5 in the Table 1), rice (number 7 in the Table 1), Bilberry (number 12 in the Table 1), and, respectively, blood test of patient 1 (number 18 in the Table 1) and their atherosclerosis pathology (number 19 in the Table 1). The effect of the latter two parameters shows that the proposed food matrix has an integral effect on the condition of two patients.

On average, only 4.12% of independent variable coefficients have a negative effect on blood test of patient 2. In other words, most products from the set have a positive effect on blood test of patient 2, or these nutrients guarantee the further development of osteoporosis.

Let's look at how positive trends of independent parameter effect on blood test of patient 2 determine their pathology, or osteoporosis. The column that specifies osteoporosis as dependent variable (number 21 in the Table 1) is increased by the nutrients contained in the following products: squash (number 2 in the Table 1), bananas (number 10 in the Table 1), condensed milk (number 12 in the Table 1), hazelnut (number 13 in the Table 1), and blood test parameters of the patient (number 21 in the Table 1) with osteoporosis. On average, nutrients and blood test of patient 2 contribute to the exacerbation of osteoporosis by 5.1%.

Therefore, matrix data show that nutrients and blood test values contribute to the development of osteoporosis in patient 2. At the same time, nutrients in other products such as red bell pepper (number 4 in the Table 1), rice (number 7 in the Table 1), half-and-half cottage cheese (number 14 in the Table 1), hazelnut (number 17 in the Table 1) and blood test of patient 2 reduce osteoporosis by 9.2% on average.

Therefore, on the one hand, some nutrients and blood test parameters are conducive to the development of osteoporosis, while others serve to reduce osteoporosis. It should be noted that conventional densitometry results showed that the spine of the patient 2 was absolutely normal but their hips joints started manifesting early signs of osteoporosis.

It is evident that the effect of nutrients and blood test parameters on the pathology is nonlinear and that reduced effect of nutrients and blood test parameters on the pathology does not always imply that pathological processes are eliminated. The most likely explanation is that the rate of pathology development is reduced, up until the development is halted. Another very important thing is that the effect of nutrients on blood parameters and vice versa is individual, as shown by the examples of atherosclerosis and osteoporosis. The same food set cannot be used to prevent a pathology for different patients.

Currently, there are tens of thousands of food products with nutrients measured biochemically and expressed as decimal weights. That is why the vital issue that was not addressed before can be actually solved. This issue is a calculation of correlations between macro and micronutrients, cell-level blood parameters, and pathology parameters. The result obtained by this method is a calculation of correlations, represented also graphically, that allows to perform targeted diagnostics and determine the cause and effect behind a number of pathologies.

We are speaking about studying and calculating correlations between nutrients, blood and pathology parameters. As long as food products, blood and pathology parameter share the same spectrum of organic and inorganic nutrients, so it will be possible to determine correlations between nutrients, blood and pathology parameters for both individual patients and a plurality of patients.

The most important goal for an individual patient is to determine which food products with a diverse combination of nutrients are excessive and prevalent, and which are deficient in terms of levels and correlations. Both excess and deficiency in nutrients for an individual patient inevitably lead to multiple pathologies.

The study of nutrients, blood and pathology parameters for a plurality of regional patients will make it possible to determine with relative precision the pathologies that may be prevalent in the region. Besides, diagnostics of correlations between nutrients, blood and pathology parameters helps identify excess and deficiency of nutrients, therefore, assisting in managing manufacture, delivery and pricing for food and medicinal products.

The most important aspect of determining the correlations of nutrients is the medical aspect. It is quite possible to determine how nutrients in food affect the blood of an individual patient.
1. The information database was created: 1000 blood tests from individual patients were digitized in accordance with the method in the U.S. Pat. No. 2,632,509.
2. The information database on food products and nutrients was created. 3000 products and 78000 constituent nutrients were digitized.
3 A technology of digitizing nutrients and calculating interaction levels and correlations between products and nutrients using statistical methods was developed. It was established that product start interacted even before their entry to the human digestive system, forming stable structures of interaction and correlation.
4. Matrix data presentation and statistical methods allowed for creating a sustainable method for selecting product sets for individual patients with diverse pathologies to eliminate the effect of these pathologies on their health.

4. The results of the studies above show that there is a real opportunity to develop computer application for selecting individual product sets for individual patients with a pathology for active prevention and elimination of the same.

5. The proposed method allows for designing an instrumental digital platform for a wide audience of persons skilled in the art. This platform could be used not only for digitizing nutrients, blood test and pathology parameters, but the data of medicinal products so that they could be included in the data matrix to evaluate and measure their effect on individual patient pathologies and ensure effective therapy.

### REFERENCES

[symbol] [symbol] [symbol] [symbol] [symbol] [symbol] [symbol] [symbol] [symbol] 1999 ( [symbol] 424-465. Sushansky A. G., Liflyandsky V. G. Healthy Food Encyclopedia. V.1. Food for Health/St Petersburg, Neva Publishing House, 1999)
2. [symbol] [symbol] [symbol] [symbol] [symbol] [symbol] [symbol] [symbol] [symbol] —No 28.—C. 26-29 (Bogatyrev A. N. Natural Food Products as Health of the Nation/Bogatyrev A. N., Pryanichnikova N. S., Makeeva I. A.//Food Industry—2017—issue 8—pp. 26-29)
3. [symbol] [symbol] [symbol] [symbol] [symbol] [symbol] [symbol] [symbol] [symbol] [symbol] [symbol] [symbol] [symbol] [symbol] [symbol] —2016.—No 6—C.59-62, (Makeeva I. A. Scientific Foundation Behind Terminology and Classification of Animal Organic Products/I. A. Makeeva, Z. Y. Belyakova, N. S. Pryanichnikova, A. N. Bogatyrev//Food Industry—2016—No 6, pp. 59-62)
[symbol] [symbol] [symbol] [symbol] [symbol] [symbol] [symbol] [symbol] [symbol] —2011.—No 1.—C. 52-55. (Bogatyrev A. N. Vitamins as Vital Components of 21^{st} Century Food Products/A. N. Bogatyrev//Meat Industry—2011—No 1—pp. 52-55)
5. [symbol] [symbol] — [symbol] / [symbol] // [symbol] 2010.—No 10.—C. 5-9 (Bogatyrev A. N. Healthy Food Means Healthy Nation/A. N. Bogatyrev//Meat Industry—2010—No 10—pp. 5-9)
6. [symbol] [symbol] [symbol] — [symbol] [symbol] / [symbol] // [symbol] -2010.—No 2.—C. 4-7 (Bogatyrev A. N. Safe Food Products as a Social Challenge/A. N. Bogatyrev//Meat Industry—2010—No 2—pp. 4-7)
7. [symbol] [symbol] [symbol] [symbol] [symbol] [symbol] [symbol] [symbol] [symbol] [symbol] [symbol] // [symbol] [symbol] —2008.—No 5.—C. 62-66. (A. N. Bogatyrev. Scientific Principles of Enriching Food Products with Micronutrients/A. N. Bogatyrev, V. M. Korotkiy, M. N. Dadashev, A. A. Kukharenko//Food Industry—2008—No 5—pp. 62-66).
8. [symbol] [symbol] [symbol] [symbol] [symbol] [symbol] / [symbol] // [symbol] [symbol] —2003—No 2.—C. 17-19. (Bogatyrev [symbol] of Ecofriendly Food/A. N. Bogatyrev//Dairy Industry—2003—No 2—pp. 17-19).
8. Oleg V. Gaisenok, Victor N. Lituev. Prospects for the application of mathematical modeling in clinical medicine. Cardiometry; Issue 14; May 2019; p.64-70; DOI: 10.12710/cardiometry.2019.14.6470; http://www.cardiometry.net/issues/no14-may-2019/mathematical-modeling-in-clinical-medicine

What is claimed is:

1. A method for generating individual nutritional recommendations for a user, performed at least by one processor and comprising the following steps:
data on at least one food product of a user are obtained;
at least one recommendation is generated for the user, wherein
parameters characterizing the user's condition are measured including complete blood count, pathologies are identified, and individual user data is converted into a numerical sequence,
each product is assigned a corresponding numerical sequence depending on the individual nutrient levels,
correlations between individual user parameters and food parameters are calculated by designing a personal matrix of each product effect on the user parameters; for this purpose, factorial weights of each individual product effect on each user parameter are calculated,
the resulting personal matrix of each product effect on the user parameters comprising factorial weights of each individual product effect on the user pathology is statistically processed using the processor; the matrix is further employed to generate recommendations for the user, wherein products with negative factorial weights are not recommended and products with positive factorial weights are recommended, for which purpose
The personal matrix is built as follows: the matrix cells are filled with values for each measured parameter divided into six ranges, namely:
i the value of the measured parameter falls within the range of permissible values for said parameter;
ii the value of the measured parameter is more than the average value of the range of permissible values
iii the value of the measured parameter is less than the average value of the range of permissible values;
iv coefficient of variation for the measured parameter falls within the range of 0 to 0.3454;
v coefficient of variation for the measured parameter falls within the range of 0.3455 to 0.8;
vi coefficient of variation for the measured parameter is more than 0.8.
